(19) United States
(12) Patent Application Publication
(10) Pub. No.: US 2025/0352680 A1
(43) Pub. Date: Nov. 20, 2025

(54) THERMAL CONTAINER TREATMENT APPARATUS AND METHOD FOR OPERATING THE THERMAL CONTAINER TREATMENT APPARATUS

(71) Applicant: Krones AG, Neutraubling (DE)

(72) Inventors: Stefan KAATZ, Fleckeby (DE); Jesse SCHMUCK, Flensburg (DE)

(21) Appl. No.: 19/208,451

(22) Filed: May 14, 2025

(30) Foreign Application Priority Data
May 15, 2024 (DE) 102024113557.3

(51) Int. Cl. A61L 2/04; A61L 2/24; B65B 55/10; B65B 57/02

(52) U.S. Cl. CPC A61L 2/04; A61L 2/24; B65B 55/10; B65B 57/02; A61L 2202/121; A61L 2202/122; A61L 2202/123; A61L 2202/14; A61L 2202/15; A61L 2202/23

(57) ABSTRACT

A thermal container treatment apparatus comprising a conveying apparatus for containers, a treatment zone with a spraying apparatus for dispensing treatment medium to containers, a tank for collecting and providing treatment medium and a line connection between the tank and the spraying apparatus, wherein a pump is provided in the line connection. A temperature sensor is provided in the tank below a filling level of the treatment medium. The thermal container treatment apparatus comprises a control apparatus which is configured to operate the pump at a rated power for dispensing treatment medium by the spraying apparatus during normal operation in the treatment zone and not to operate the pump during standby operation in the treatment zone or to operate the pump at a reduced rated power without treatment medium being able to be dispensed by the spraying apparatus.

### CROSS-REFERENCE TO RELATED APPLICATION(S) AND CLAIM OF PRIORITY

This application claims priority to German Patent Application No. 102024113557.3, filed May 15, 2024, the disclosure of which is incorporated herein by reference in its entirety.

### TECHNICAL FIELD

The present invention relates to a thermal container treatment apparatus and to a method for operating the thermal container treatment apparatus according to the independent claims.

### BACKGROUND

Thermal container treatment apparatuses, such as coolers, warmers or pasteurizers, are known in which containers in different, consecutive treatment zones can be sprayed with treatment medium dispensed by spraying apparatuses. The dispensing of the treatment medium by the spraying apparatuses takes place in a waiting or preparatory operating mode (standby mode) as well as in a normal operating mode of the thermal container treatment apparatuses. This can be considered unfavorable in terms of electrical and thermal energy consumption.

The object of the invention is to provide a thermal container treatment apparatus and a method for operating the thermal container treatment apparatus, which can enable a saving of electrical and thermal energy consumption and at the same time also an immediate operational readiness of the thermal container treatment apparatus.

### SUMMARY

The object is achieved by the thermal container treatment apparatus and the method for operating the thermal container treatment apparatus according to the independent claims. Further embodiments are disclosed in the dependent claims.

The thermal container treatment apparatus according to the invention, such as a cooler, a warmer or a pasteurizer, comprises a conveying apparatus for containers, a treatment zone with a spraying apparatus for dispensing treatment medium to containers, a tank for collecting and providing treatment medium and a line connection between the tank and the spraying apparatus, wherein a pump is provided in the line connection. A temperature sensor is provided to measure the temperature of the treatment medium. The thermal container treatment apparatus further comprises a control apparatus which is configured to operate the pump at a rated power for dispensing treatment medium by the spraying apparatus during normal operation in the treatment zone and not to operate the pump during standby operation in the treatment zone or to operate the pump at a reduced rated power without treatment medium being able to be dispensed by the spraying apparatus.

By not operating the pump or by operating the pump at a reduced rated power, thermal energy consumption can be reduced, since there is no atomization of the treatment medium when it is dispensed using the spraying apparatus. The electrical energy consumption of the pump can also be reduced.

The temperature of the treatment medium in the tank can be measured and monitored by the temperature sensor. For example, the operational readiness of the thermal container treatment apparatus after a standby operation can be ensured without major delays.

Failure to operate the pump during standby operation can involve the control apparatus turning the pump off from its rated power. To return to normal operation, the control apparatus can switch the pump back on and operate it at rated power.

The line connection can end in the tank with an intake connection and the temperature sensor can be arranged adjacent to the intake connection.

A bypass can be provided between the line connection and the tank, wherein the bypass can end above the filling level of the treatment medium in the tank. For example, the pump bypass can be arranged downstream. For example, a valve can be provided in the line connection between the pump and the spraying apparatus. For example, the bypass can be provided as a pipe with a diameter of less than 50 millimeters.

When operating the pump at a reduced rated power during standby operation, the bypass and the valve in the line connection can be used, for example. The valve can be provided in such a way that treatment medium which can be pumped out of the tank by the pump operated at reduced rated power cannot pass through the valve but can be diverted into the bypass. The amount of “reduced performance” may depend on the valve used and/or the diameter of the bypass.

However, when the pump is operated at its rated power, the treatment medium can pass through the valve. Diverting treatment medium into the bypass can be minimal or completely prevented when the pump is operated at rated power.

The line connection can also have a connection to at least one temperature control valve, wherein the at least one temperature control valve can be connected to a heating and/or cooling system for providing temperature-controlled treatment medium.

The at least one temperature control valve can be connected to the line connection between an outlet of the line connection from the tank and the pump.

The control apparatus can be further configured to control a temperature of the treatment medium in the tank by controlling the at least one temperature control valve.

The thermal container treatment apparatus can comprise a plurality of the treatment zones, wherein the treatment zones can be configured as warming zones or as cooling zones. For example, several consecutive cooling zones can be provided for a cooler. For example, several consecutive warming zones can be provided for a warmer.

The thermal container treatment apparatus can comprise a plurality of the treatment zones, wherein at least one of them can be configured as a warming zone, at least one of them can be configured as a pasteurization zone and at least one of them can be configured as a cooling zone. The at least one warming zone and the at least one cooling zone can be configured together as a recuperation stage. A first line connection can be provided from a tank of a warming zone to a spraying apparatus of a cooling zone and a second line connection can be provided from a tank of a cooling zone to a spraying apparatus of a warming zone. A third line connection can be provided from a tank of a pasteurization zone to the spraying apparatus of this pasteurization zone.

The control apparatus can be further configured to not operate any of pumps during a standby operation of the thermal container treatment apparatus or to operate all pumps at a reduced rated power so as not to dispense treatment medium by the respective spraying apparatus.

The control apparatus can be further configured, after a standby operation of the thermal container treatment apparatus in order to achieve normal operation, at a first given point in time before a planned supply of containers to the at least one warming zone, to operate the pumps of the at least one warming zone and the at least one cooling zone at the rated power to dispense treatment medium from the spraying apparatuses of the at least one warming zone and the at least one cooling zone, and then at a second given point in time before a planned supply of containers from the at least one warming zone to the at least one pasteurization zone, to operate the pump of the at least one pasteurization zone at the rated power to dispense treatment medium from the spraying apparatus of the at least one pasteurization zone.

After normal operation of the thermal treatment plant has been reached, the control apparatus can be further configured to achieve a standby operation, after containers have left the at least one pasteurization zone, not to operate the pump of the at least one pasteurization zone or to operate the pump of the at least one pasteurization zone at a reduced rated power so as not to dispense treatment medium by the spraying apparatus of the at least one pasteurization zone, and then, after containers have left the last of the at least one cooling zone, not to operate any of the pumps of the at least one warming zone and the at least one cooling zone or to operate all pumps of the at least one warming zone and the at least one cooling zone at a reduced rated power so as not to dispense treatment medium by the respective spraying apparatuses.

The temperature sensor can be provided in the tank below a filling level of the treatment medium, or the temperature sensor can be provided in a return line. The return line can lead from the tank to a heating system and/or a cooling system. The cooling system can be configured as a first heat exchanger, which can be line-connected to the first temperature control valve. The heating system can be configured as a second heat exchanger, which can be line-connected to the second temperature control valve.

A method for controlling a thermal container treatment apparatus as described above or further below is provided.

The method can comprise, during standby operation in the treatment zone, not operating the pump or operating the pump at a reduced rated power without dispensing treatment medium by the spraying apparatus.

The method can comprise, during normal operation in the treatment zone, operating the pump at a rated power to dispense treatment medium by the spraying apparatus.

For a thermal container treatment apparatus comprising at least one temperature control valve, the method can comprise controlling the at least one temperature control valve to control a temperature of the treatment medium in the tank.

For a thermal container treatment apparatus comprising a plurality of the treatment zones, wherein at least one of them is configured as a warming zone, at least one of them is configured as a pasteurization zone and at least one of them is configured as a cooling zone, wherein the at least one warming zone and the at least one cooling zone together are configured as a recuperation stage, wherein a first line connection is provided from a tank of a warming zone to a spraying apparatus of a cooling zone and a second line connection is provided from a tank of a cooling zone to a spraying apparatus of a warming zone, and wherein a third line connection is provided from a tank of a pasteurization zone to the spraying apparatus of this pasteurization zone, the method can comprise, during standby operation of the thermal container treatment apparatus, not operating any of the pumps or operating all pumps at a reduced rated power so as not to dispense treatment medium by the respective spraying apparatus.

To achieve normal operation of the thermal container treatment apparatus after a standby operation of the thermal container treatment apparatus, the method can comprise: at a first given point in time before a planned supply of containers to the at least one warming zone, operating the pumps of the at least one warming zone and the at least one cooling zone at the rated power to dispense treatment medium from the spraying apparatuses of the at least one warming zone and the at least one cooling zone, and then at a second given point in time before a planned supply of containers from the at least one warming zone to the at least one pasteurization zone, operating the pump of the at least one pasteurization zone at the rated power to dispense treatment medium from the spraying apparatus of the at least one pasteurization zone.

The method can comprise, in order to achieve standby operation of the thermal treatment plant after normal operation of the thermal treatment plant has been achieved: after containers have left the at least one pasteurization zone, not operating the pump of the at least one pasteurization zone or operating the pump of the at least one pasteurization zone at a reduced rated power so as not to dispense treatment medium by the spraying apparatus of the at least one pasteurization zone, and then, after containers have left the last of the at least one cooling zone, not operating any pumps of the at least one warming zone and the at least one cooling zone or operating all pumps of the at least one warming zone and the at least one cooling zone at a reduced rated power so as not to dispense treatment medium by the respective spraying apparatuses

Other technical features may be readily apparent to one skilled in the art from the following figures, descriptions, and claims.

### BRIEF DESCRIPTION OF THE DRAWINGS

For a more complete understanding of this disclosure and its advantages, reference is now made to the following description, taken in conjunction with the accompanying drawings, in which:

FIG. 1 shows a schematic view of a treatment zone in normal operation;

FIG. 2 shows the treatment zone of FIG. 1 in standby operation;

FIG. 3 shows a schematic view of two treatment zones connected as a recuperation stage in normal operation;

FIG. 4 shows the two treatment zones of FIG. 3 in standby operation;

FIG. 5 shows a schematic view of a pasteurizer with warming, pasteurization and cooling zones in normal operation;

FIG. 6 shows the pasteurizer of FIG. 5 in standby operation;

FIG. 7 shows the pasteurizer of FIG. 5 during the inflow of containers and;

FIG. 8 shows the pasteurizer of FIG. 5 during discharge of containers.

### DETAILED DESCRIPTION

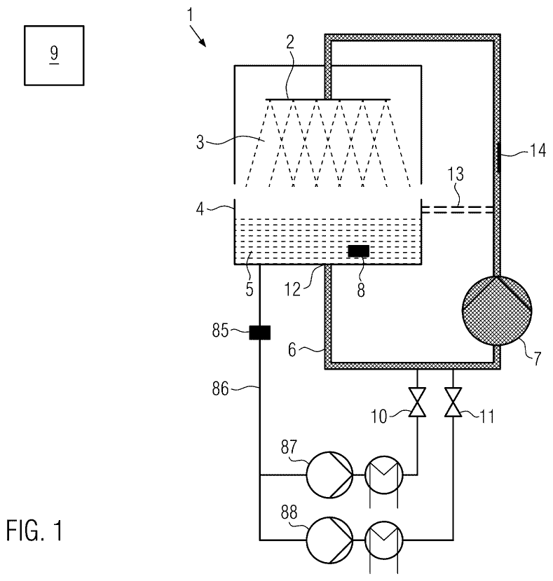

FIG. 1 shows a schematic view of a treatment zone 1 in normal operation. The treatment zone 1 can be comprised by a thermal container treatment apparatus, such as a cooler, a warmer or a pasteurizer. For example, this treatment zone 1 can be used as a pasteurization zone.

The treatment zone 1 comprises a spraying apparatus 2 for dispensing treatment medium 3, for example onto containers that can be transported through the treatment zone 1. For example, the spraying apparatus 2 can comprise nozzles or other dispensing means by which the treatment medium 3 can be dispensed.

The treatment medium 3 dispensed by the spraying apparatus 2 reaches at least in part a tank 4 which is arranged below the spraying apparatus 2 (for example, viewed in a direction of gravity) to collect the dispensed treatment medium 3 and to provide treatment medium 5.

A line connection 6 is provided between the tank 4 and the spraying apparatus 2, wherein a pump 7 is provided in the line connection 6, which pump is operated, for example, at a rated power, so that treatment medium 5 can be transported from the tank 4 and through the line connection 6 to the spraying apparatus 2 and dispensed there. The line connection 6 can be completely or substantially completely filled with treatment medium 5, as schematically indicated by the hatching in the line connection 6.

A temperature sensor 8 is provided in the tank 4 below a filling level of the treatment medium 5. It can be used to measure the temperature of the treatment medium 5 in the tank 4. Alternatively, a temperature sensor 85 can be provided in a return line 86 for measuring the temperature of the treatment medium. A value of the measured temperature can be transmitted to a control apparatus 9.

For controlling the temperature of treatment medium, a first temperature control valve 10 is connected to a cooling system and a second temperature control valve 11 is connected to a heating system, wherein the first and second temperature control valves 10, 11 are also connected to the line connection 6. Targeted temperature control can be carried out, for example in addition to a heat or cold transfer into the dispensed treatment medium 3 upon contact with the containers. The temperature control valves 10, 11 can be connected to the line connection 6 between an outlet 12 of the tank 4 and the pump 7.

The cooling system can be a first heat exchanger 87 arranged in the return line 86 upstream of the first temperature control valve 10. The heating system can be a second heat exchanger 88 arranged in the return line 86 upstream of the second temperature control valve 11.

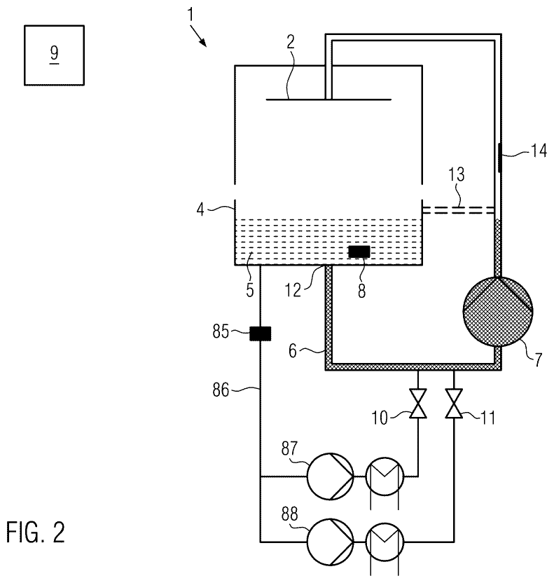

Optionally, a bypass 13 can be provided between the line connection 6 and the tank 4, wherein the bypass 13 can end above the filling level of the treatment medium 5 in the tank 4. The bypass 13 of the pump 7 can be arranged downstream. For example, the bypass 13 can be provided as a pipe with a diameter of less than 50 millimeters. Optionally, a valve 14 can also be provided in the line connection 6 between the pump 7 and the spraying apparatus 2. The functioning of the bypass 13 and the valve 14 is explained in greater detail in conjunction with FIG. 2.

The control apparatus 9 is configured to operate the pump 7 during normal operation, for example at the rated power. Furthermore, the control apparatus 9 is configured to be able to introduce warm and/or cool treatment medium into the line connection 6 by controlling the temperature control valves 10, 11. In this way, the temperature of the treatment medium 5 in the tank 4 can be indirectly controlled.

FIG. 2 shows the treatment zone of FIG. 1 in standby operation. The control apparatus 9 is further configured to not operate the pump 7 during standby operation and to stop it accordingly during the transition from normal operation to standby operation. When the pump 7 is not in operation, no treatment medium is dispensed by the spraying apparatus 2. Since the pump 7 no longer pumps the treatment medium in the line connection 6, the line connection 6 can only be partially filled with treatment medium, as schematically indicated by the hatching in the line connection 6. The filling of the line connection 6 extends from the tank 4 to a height corresponding to the filling level of the treatment medium 5 in the tank 4.

Standby operation can be started when there are no more containers in treatment zone 1.

By the temperature sensor 8, the temperature of the treatment medium 5 in the tank 4 can be measured and thus monitored, for example, or alternatively, the temperature of the treatment medium in the return line 86 can be measured and monitored by the temperature sensor 85. If the measured temperature falls below a predetermined setpoint, the second temperature control valve 11 can be controlled by the control apparatus 9 and warm treatment medium can be introduced into the line connection 6. If the measured temperature rises above a predetermined setpoint, the first temperature control valve 10 can be controlled by the control apparatus 9 and cool treatment medium can be introduced into the line connection 6.

As an alternative to not operating the pump 7 during standby operation, the control apparatus 9 can operate the pump 7 at a reduced rated power. Then the bypass 13 and the valve 14 in the line connection 6, which were previously specified as optional, can be used. The valve 14 is provided in such a way that treatment medium which can be pumped out of the tank 4 by the pump 7 operated at reduced power cannot pass through the valve 14 but can be diverted into the bypass 13. The amount of the reduced rated power can depend on the valve 14 used and/or a diameter of the bypass 13.

When the pump 7 is operated at its rated power, the treatment medium can pass through the valve 14. Diverting treatment medium into the bypass 13 can be minimal or completely prevented when the pump 7 is operated at rated power.

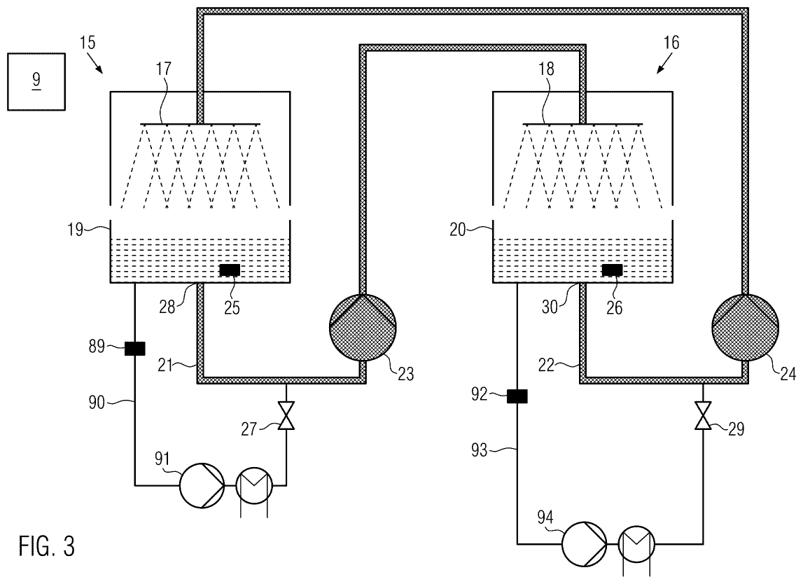

FIG. 3 shows a schematic view of two treatment zones 15, 16 connected as a recuperation stage in normal operation. The first treatment zone 15 can be a warming zone, for example a first warming zone of a pasteurizer. The second treatment zone 16 can be a cooling zone, for example a final warming zone of a pasteurizer.

The first and second treatment zones 15, 16 each comprise a spraying apparatus 17, 18 for dispensing treatment medium. For example, the spraying apparatuses 17, 18 can comprise nozzles or other dispensing means by which the treatment medium can be dispensed.

In each of the first and second treatment zones 15, 16, the treatment medium dispensed by the respective spraying apparatus 17, 18 reaches at least partly a tank 19, 20 of this treatment zone, which tank is arranged below the respective spraying apparatus 17, 18 (for example, viewed in a direction of gravity) of this treatment apparatus 15, 16 to collect the dispensed treatment medium and to provide treatment medium.

The tank 19 of the first treatment zone 15 is connected to the spraying apparatus 18 of the second treatment zone 16 by a first line connection 21 and the tank 20 of the second treatment zone 16 is connected to the spraying apparatus 17 of the first treatment zone 15 by a second line connection 22.

In the first line connection 21, a first pump 23 is provided, which is operated, for example, at a rated power by the control apparatus 9, so that treatment medium can be transported from the tank 19 of the first treatment zone 15 and through the first line connection 21 to the spraying apparatus 18 of the second treatment zone 16 and dispensed there. The first line connection 21 can be completely or substantially completely filled with treatment medium.

In the second line connection 22, a second pump 24 is provided, which is operated, for example, at a rated power by the control apparatus 9, so that treatment medium can be transported from the tank 20 of the second treatment zone 16 and through the second line connection 22 to the spraying apparatus 17 of the first treatment zone 15 and dispensed there. The second line connection 22 can be completely or substantially completely filled with treatment medium.

In the tanks 19, 20 of the first and second treatment zones 15, 16, a temperature sensor 25, 26 is provided below a filling level of the treatment medium. It can be used to measure the temperature of the treatment medium in the corresponding tank 19, 20. Alternatively, a temperature sensor 89, 92 can be provided in each of the return lines 90, 93 for measuring the temperature of the treatment medium. A respective value of the respective measured temperature can be transmitted to the control apparatus 9.

For temperature control of the treatment medium in the tank 19 of the first treatment zone 15, a first temperature control valve 27 is connected to a cooling system and the first line connection 21. Targeted temperature control can be carried out, for example in addition to a heat or cold transfer into the dispensed treatment medium upon contact with the containers. The first temperature control valve 27 can be connected to the first line connection 21 between an outlet 28 from the tank 19 of the first treatment zone 15 and the first pump 23.

For temperature control of the treatment medium in the tank 20 of the second treatment zone 16, a second temperature control valve 29 is connected to a heating system and the second line connection 22. Targeted temperature control can be carried out, for example in addition to a heat or cold transfer into the dispensed treatment medium upon contact with the containers. The second temperature control valve 29 can be connected to the second line connection 22 between an outlet 30 from the tank 20 of the second treatment zone 16 and the second pump 24.

The cooling system can be a first heat exchanger 91 arranged in the return line 90 upstream of the first temperature control valve 27. The heating system can be a second heat exchanger 94 arranged in the return line 93 upstream of the second temperature control valve 29.

The control apparatus 9 is configured to operate the pumps 23, 24 during normal operation, for example at the rated power, so that treatment medium is dispensed by the spraying apparatuses 17, 18 of the first and second treatment zones 15, 16. Furthermore, the control apparatus 9 is configured to be able to introduce cool treatment medium into the first line connection 21 by controlling the first temperature control valve 27 of the first treatment zone 15 and to introduce warm treatment medium into the second line connection 22 by controlling the second temperature control valve 29 of the second treatment zone 16. In this way, the temperature of the treatment medium in the tank 19, 20 of the first and second treatment zones 15, 16 can be indirectly controlled.

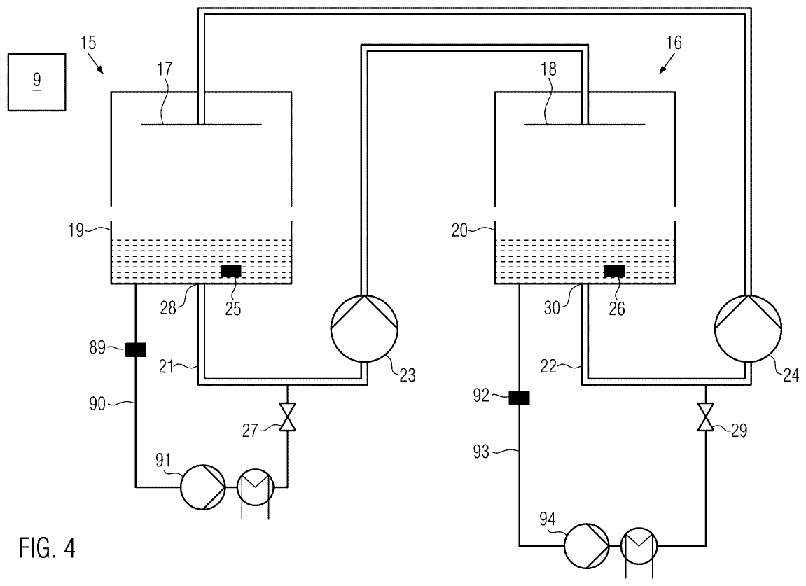

FIG. 4 shows the two treatment zones 15, 16 of FIG. 3 in standby operation. The first and second pumps 23, 24 are not operated by the control apparatus 9, so that no treatment medium is dispensed by the spraying apparatuses 17, 18 of the first and second treatment zones 15, 16. The first temperature control valve 27 of the first treatment zone 15 and the second temperature control valve 29 of the second treatment zone 16 are further controlled by the control apparatus 9.

By the temperature sensor 25 in the tank 19 of the first treatment zone 15, the temperature of the treatment medium in this tank 19 can be measured and thus monitored, for example, or alternatively, the temperature of the treatment medium in the return line 90 can be measured and monitored by the temperature sensor 89. If the measured temperature rises above a predetermined setpoint, the first temperature control valve 27 of the first treatment zone 15 can be controlled by the control apparatus 9 and cool treatment medium can be introduced into the first line connection 21.

By the temperature sensor 26 in the tank 20 of the second treatment zone 16, the temperature of the treatment medium in this tank 20 can be measured and thus monitored, for example, or alternatively, the temperature of the treatment medium in the return line 93 can be measured and monitored by the temperature sensor 92. If the measured temperature falls below a predetermined setpoint, the second temperature control valve 29 of the second treatment zone 16 can be controlled by the control apparatus 9 and warm treatment medium can be introduced into the second line connection 22.

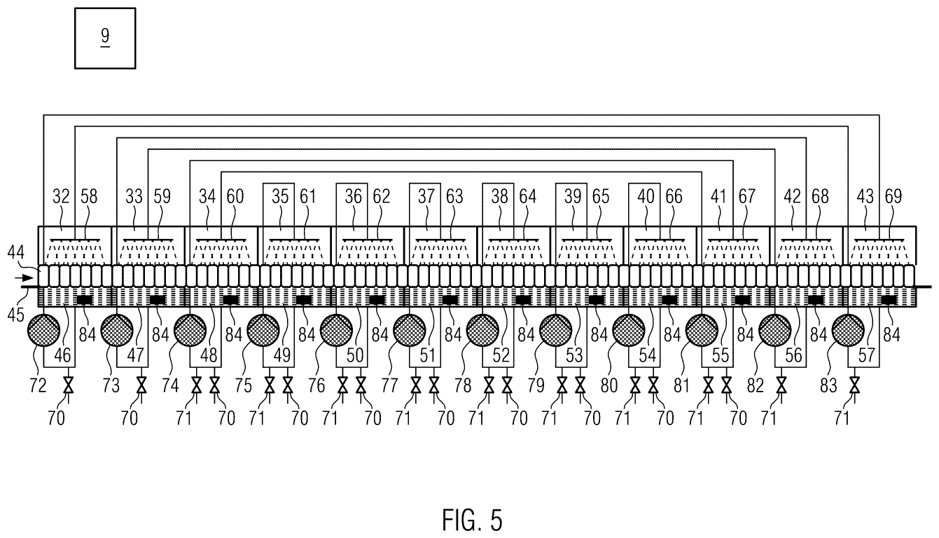

FIG. 5 shows a schematic view of a pasteurizer 31 (as a thermal container treatment apparatus) with three warming zones 32, 33, 34, six pasteurization zones 35, 36, 37, 38, 39, 40 and three cooling zones 41, 42, 43 in normal operation. Containers 44 are guided, for example on a conveyor belt 45, through the pasteurizer 31 and sprayed with treatment medium in the various zones 32-43. The containers 44 can successively pass through the first, second, third warming zones 32-34, the first, second, third, fourth, fifth and sixth pasteurization zones 35-40, the first, second and third cooling zones 41-43. What is described here and below can also apply to a pasteurizer with fewer or more warming and cooling zones and with fewer or more pasteurization zones.

The first warming zone 32 and the third cooling zone 43, the second warming zone 33 and the second cooling zone 42 as well as the third warming zone 34 and the first cooling zone 43 are each operated as a recuperation stage. The tank 46 of the first warming zone 32 is line-connected to the spraying apparatus 69 of the third cooling zone 43 and the tank 57 of the third cooling zone 43 is line-connected to the spraying apparatus 58 of the first warming zone 32. The tank 47 of the second warming zone 33 is line-connected to the spraying apparatus 68 of the second cooling zone 42 and the tank 56 of the second cooling zone 42 is line-connected to the spraying apparatus 59 of the second warming zone 33. The tank 48 of the third warming zone 34 is line-connected by line to the spraying apparatus 67 of the first cooling zone 41 and the tank 55 of the first cooling zone 41 is line-connected to the spraying apparatus 60 of the third warming zone 34.

In the six pasteurization zones 35-40, a tank 49-54 of one of the pasteurization zones 35-40 is line-connected to the spraying apparatus 61-66 of the same pasteurization zone 35-40.

A temperature sensor 84 is arranged in each of the tanks 46-57, with which the temperature of the treatment medium present in the tank 46-57 can be measured and monitored. Alternatively, instead of the temperature sensors 84 in the tanks, a temperature sensor (not shown) can be provided in the return line.

The first and second warming zones 32, 33 each comprise a second temperature control valve 70 which is connected to a heating system.

The third warming zone 34, the six pasteurization zones 35-40 and the first cooling zone 41 each comprise a first temperature control valve 71 connected to a cooling system and a second temperature control valve 70 connected to a heating system.

The second and third cooling zones 42, 43 each comprise a first temperature control valve 71 which is connected to a cooling system.

During normal operation, the respective pump 72-83 is operated by all treatment zones 32-43 and the first and second temperature control valves 71, 70 are controlled respectively.

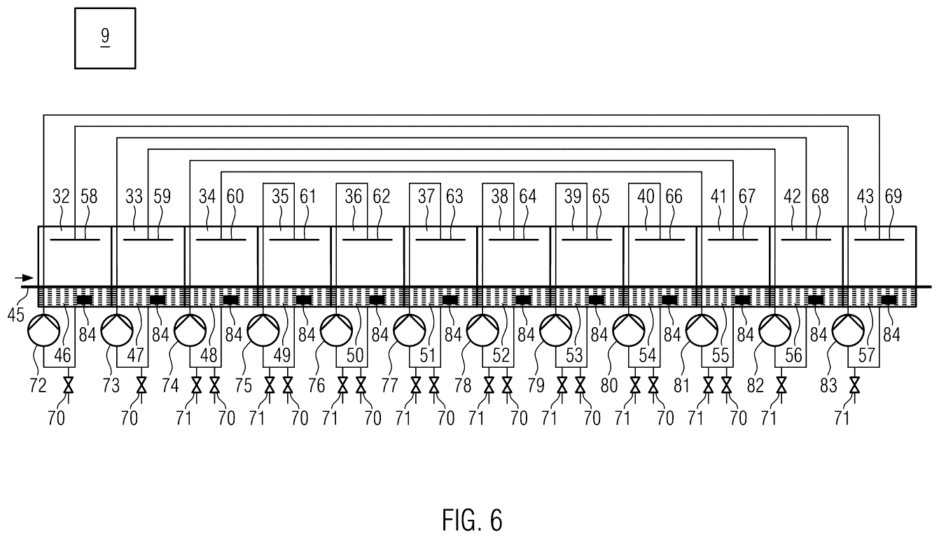

FIG. 6 shows the pasteurizer of FIG. 5 in standby operation. Here, all treatment zones 32-43 are in standby operation, which is why it can also be said that the pasteurizer 31 is in standby operation. In standby operation, none of the pumps 72-83 are operated, but the first and second temperature control valves 71, 70 continue to be controlled in order to be able to maintain the temperature of the treatment medium in the respective tanks 46-57 at a setpoint or within a setpoint temperature range if the spraying apparatuses 58-69 no longer dispense any treatment medium that can reach the respective tanks 46-57. The standby operation of all treatment zones 32-43 of the pasteurizer 31 can be provided when there are no containers for treatment in the pasteurizer 31. The normal operation of the pasteurizer 31 shown in FIG. 5 can be terminated by the control apparatus 9, after the last container has left the pasteurizer 31, by stopping the pumps 72-83 and then not operating the pumps 72-83 in order to enter standby operation.

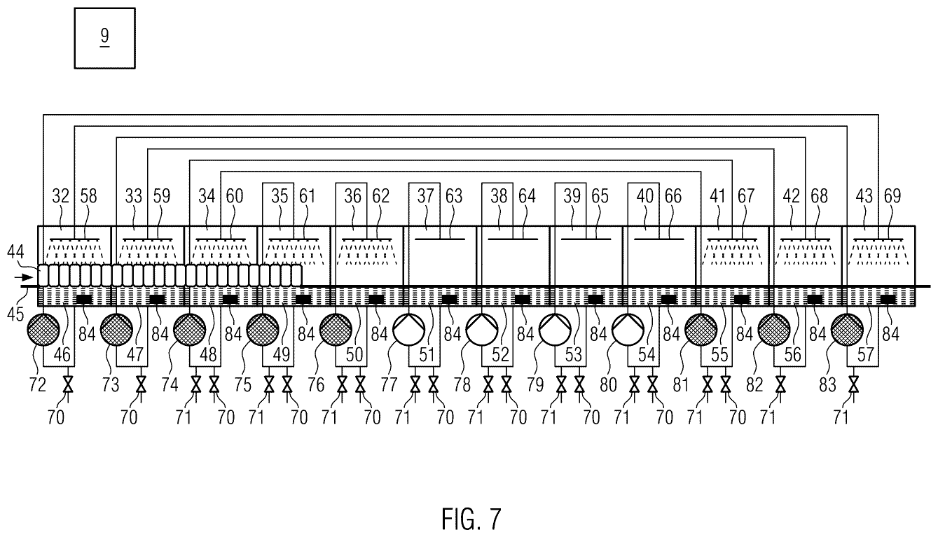

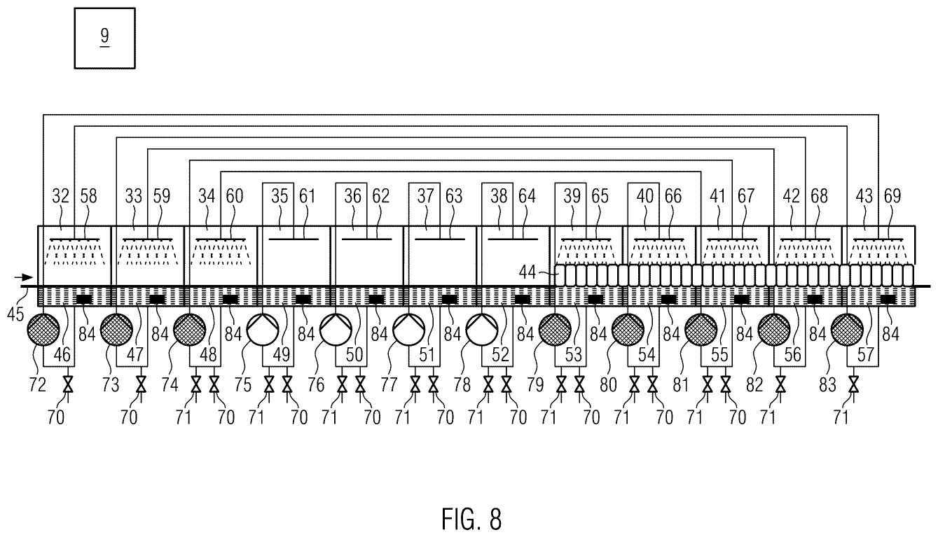

In conjunction with FIGS. 7 and 8, aspects are explained as to how individual pasteurization zones 35-40 and/or recuperation stages can be brought from standby operation into normal operation or from normal operation into standby operation, for example taking into account a point in time at which containers 44 are introduced into the pasteurizer 31 and/or taking into account a point in time when containers 44 will arrive at a specific treatment zone 32-43.

FIG. 7 shows the pasteurizer 31 of FIG. 5 during the introduction of containers 44, wherein at the point in time shown, containers 44 are already in the first to third warming zones 32-34 and some containers 44 are in the first pasteurization zone 35. The illustrated running-in of the containers 44 can take place following a standby operation of the pasteurizer 31, as shown in FIG. 6.

For example, by the control apparatus, the pump 72 of the first warming zone 32 and the pump 83 of the third cooling zone 43 were put into operation at a first point in time preceding a planned supply of containers 44 (the supply point in time can be known) after a standby operation of the pasteurizer 31. The time period that elapses between the start-up of the pumps 72, 83 and the arrival of the first container 44 in the first warming zone 32 can be regarded as a lead time for a self-adjustment control of the recuperation stage from the first warming zone 32 and the third cooling zone 43. It can thus be possible for the control of the recuperation stage from the first warming zone 32 and the third cooling zone 43 to adjust to the upcoming treatment of the containers 44 with treatment medium for normal operation during this lead time. By operating the pump 72 of the first warming zone 32 and the pump 83 of the third cooling zone 43, the spraying apparatus 58 of the first warming zone 32 and the spraying apparatus 69 of the third cooling zone 43 dispense treatment medium, which can be applied to the containers 44 present in the first warming zone 32.

Such a lead time can also be provided for the further warming zones 33, 34 and the pasteurization zones 35-40. Lead times of the same or different lengths can be provided for different zones.

The second warming zone 33 following the first warming zone 32 was put into normal operation together with the second cooling zone 42 with a time delay from the first point in time, for example at a second point in time, by the pump 72 of the second warming zone 33 and the pump 82 of the second cooling zone 42 being put into operation at the second point in time by the control apparatus 9. The second point in time can be determined, for example, taking into account a known or estimated transport time of the containers 44 through the first warming zone 33, i.e., until a first container 44 from the first warming zone 32 can arrive at the second warming zone 33, and taking into account a desired lead time. By operating the pump 73 of the second warming zone 33 and the pump 82 of the second cooling zone 42, the spraying apparatus 59 of the second warming zone 33 and the spraying apparatus 68 of the second cooling zone 42 dispense treatment medium, which can be applied to the containers 44 in the second warming zone 33.

The third warming zone 34 following the second warming zone 33 was put into normal operation together with the first cooling zone 41 with a time delay from the second point in time, for example at a third point in time, by the pump 74 of the third warming zone 34 and the pump 81 of the first cooling zone 41 being put into operation at the third point in time by the control apparatus 9. The third point in time can be determined, for example, taking into account a known or estimated transport time of the containers 44 through the second warming zone 33, i.e., until a first container 44 from the second warming zone 33 can arrive at the third warming zone 34, and taking into account a desired lead time. By operating the pump 74 of the third warming zone 34 and the pump 81 of the first cooling zone 41, the spraying apparatus 60 of the third warming zone 34 and the spraying apparatus 67 of the first cooling zone 41 dispense treatment medium, which can be applied to the containers 44 in the third warming zone 34.

The first pasteurization zone 35 following the third warming zone 34 was put into normal operation with a time delay from the third point in time, for example from a fourth point in time, by the pump 75 of the first pasteurization zone 35 being put into operation at the fourth point in time by the control apparatus 9. The fourth point in time can be determined, for example, taking into account a known or estimated transport time of the containers 44 through the third warming zone 34, i.e., until a first container 44 from the third warming zone 34 can arrive at the first pasteurization zone 35, and taking into account a desired lead time. By operating the pump 75 of the first pasteurization zone 35, treatment medium is dispensed through the spraying apparatus 61 of the first pasteurization zone 35 and applied to the containers 44.

The time period that elapses between the start-up of the pump 75 of the first pasteurization zone 35 and the arrival of the first container 44 in the first pasteurization zone 35 can be regarded here as a lead time for a self-adjustment control of the first pasteurization zone 35. It can thus be possible for the control of the first pasteurization zone 35 to adjust to the upcoming treatment of the containers 44 with treatment medium for normal operation during this lead time.

In the illustration of FIG. 7, containers 44 are located in the first, second and third warming zones 32-34 and containers 44 have only partially passed through the first pasteurization zone 35.

The second pasteurization zone 36 has also already been put into normal operation by the control apparatus 9 starting the pump 76 of the second pasteurization zone 36 so that treatment medium can be dispensed by the spraying apparatus 62 of the second pasteurization zone 36. The second pasteurization zone 36 following the first pasteurization zone 35 was put into normal operation with a time delay from the fourth point in time, for example from a fifth point in time, by the pump 76 being put into operation as described by the control apparatus 9. The fifth point in time can be determined, for example, taking into account a known or estimated transport time of the containers 44 through the first pasteurization zone 35, i.e., until a first container 44 from the first pasteurization zone 35 can arrive at the second pasteurization zone 36, and taking into account a desired lead time.

For putting into operation, the pumps 72-76 and 81-83 can each be operated at their rated power.

The third, fourth, fifth and sixth pasteurization zones 37-40 are each still in standby operation and can also be put into normal operation according to the description for the first and second pasteurization zones 35, 36.

FIG. 8 shows the pasteurizer 31 of FIG. 5 during discharge of containers 44. Containers 44 are shown only in the fifth and sixth pasteurization zones 39, 40 and in the three cooling zones 41-43.

The first, second, third and fourth pasteurization zones 35-38 are already in standby operation. By the control apparatus 9, the pumps 75-78 of these pasteurization zones 35-38 were no longer operated one after the other as soon as the containers 44 had left a corresponding pasteurization zone 35-38, so that accordingly no more treatment medium was dispensed by the corresponding spraying apparatus 61-64.

The first, second and third warming zones 32-34 continue to be in normal operation, since they each form a recuperation stage together with the third, second and first cooling zones 43, 42, 41 and containers 44 are still located in the first, second and third cooling zones 41-43.

When the containers 44 have left the fifth pasteurization zone 39, this can be put into standby operation and when the containers 44 have left the sixth pasteurization zone 40, this can then be put into standby operation.

When the containers 44 have left the first cooling zone 41, the first cooling zone 41 and the third warming zone 34 can be put into standby operation.

When the containers 44 have left the second cooling zone 42, the second cooling zone 42 and the second warming zone 33 can be put into standby operation.

When the containers 44 have left the third cooling zone 43, the third cooling zone 43 and the first warming zone 32 can be put into standby operation.

The pasteurizer 31 is then back in standby operation, as shown, for example, in FIG. 6.

While the invention has been described in relation to specific embodiments for the purpose of full and clear disclosure, the appended claims are not to be limited in this way, but are to be construed to embody all modifications and alternative structures that fall within the scope of protection of the claims and which may reasonably occur to those skilled in the art. In addition, the features of different implementation embodiments may be combined to form further embodiments of the invention.

What is claimed is:

1. A thermal container treatment apparatus, comprising:
a conveying apparatus for containers;
a treatment zone with a spraying apparatus for dispensing treatment medium to containers;
a tank for collecting and providing the treatment medium;
a line connection between the tank and the spraying apparatus, wherein a pump is provided in the line connection;
a temperature sensor provided for measuring a temperature of the treatment medium; and
a control apparatus configured, during normal operation in the treatment zone, to operate the pump at a rated power for dispensing the treatment medium by the spraying apparatus, and, during standby operation in the treatment zone, not to operate the pump or to operate the pump at a reduced rated power without the treatment medium being able to be dispensed by the spraying apparatus.

2. The thermal container treatment apparatus of claim 1, wherein the line connection in the tank ends with an intake connection, and wherein the temperature sensor is arranged adjacent to the intake connection, and/or
wherein a bypass is provided between the line connection and the tank, wherein the bypass ends above a filling level of the treatment medium in the tank, wherein the bypass is arranged downstream of the pump, wherein a valve is provided in the line connection between the pump and the spraying apparatus, wherein the bypass is provided as a pipe with a diameter of less than 50 millimeters.

3. The thermal container treatment apparatus of claim 2, wherein the line connection further comprises a connection to at least one temperature control valve, wherein the at least one temperature control valve is connected to a heating and/or cooling system for providing temperature-controlled treatment medium.

4. The thermal container treatment apparatus of claim 1, wherein the line connection further comprises a connection to at least one temperature control valve, wherein the at least one temperature control valve is connected to a heating and/or cooling system for providing temperature-controlled treatment medium.

5. The thermal container treatment apparatus of claim 4, wherein the control apparatus is further configured to control a temperature of the treatment medium in the tank by controlling the at least one temperature control valve.

6. The thermal container treatment apparatus of one of claim 5, further comprising a plurality of the treatment zones, wherein the plurality of treatment zones is configured as warming zones or as cooling zones.

7. The thermal container treatment apparatus of one of claim 1, further comprising a plurality of the treatment zones, wherein the plurality of treatment zones is configured as warming zones or as cooling zones.

8. The thermal container treatment apparatus of claim 7, wherein the control apparatus is further configured to not operate any pumps during the standby operation of the thermal container treatment apparatus or to operate all pumps at the reduced rated power so as not to dispense the treatment medium by the respective spraying apparatus.

9. The thermal container treatment apparatus of one of claim 1, further comprising a plurality of the treatment zones,
wherein at least one of the plurality of treatment zones is configured as a warming zone, at least one of the plurality of treatment zones is configured as a pasteurization zone and at least one of the plurality of treatment zones is configured as a cooling zone,
wherein at least one warming zone and at least one cooling zone are formed together as a recuperation stage, and
wherein a first line connection is provided from a tank of a warming zone to a spraying apparatus of a cooling zone, wherein a second line connection is provided from a tank of a cooling zone to a spraying apparatus of a warming zone and a third line connection is provided from a tank of a pasteurization zone to the spraying apparatus of this pasteurization zone.

10. The thermal container treatment apparatus of claim 9, wherein the control apparatus is further configured to not operate any pumps during the standby operation of the thermal container treatment apparatus or to operate all pumps at the reduced rated power so as not to dispense the treatment medium by the respective spraying apparatus.

11. The thermal container treatment apparatus of claim 10, wherein the control apparatus is further configured after the standby operation of the thermal container treatment apparatus in order to achieve the normal operation:
at a first given point in time before a planned supply of containers to the at least one warming zone, to operate the pumps of the at least one warming zone and the at least one cooling zone at the rated power for dispensing the treatment medium from spraying apparatuses of the at least one warming zone and the at least one cooling zone, and, thereafter,
at a second given point in time before the planned supply of containers from the at least one warming zone to at least one pasteurization zone, to operate a pump of the at least one pasteurization zone at the rated power for dispensing the treatment medium from the spraying apparatus of the at least one pasteurization zone.

12. The thermal container treatment apparatus of claim 11, wherein the control apparatus, in order to achieve the standby operation after reaching the normal operation of a thermal treatment plant, is further configured:
after containers have left the at least one pasteurization zone, not to operate the pump of the at least one pasteurization zone or to operate the pump of the at least one pasteurization zone at the reduced rated power so as not to dispense the treatment medium by the spraying apparatus of the at least one pasteurization zone, and thereafter
after containers have left the last of the at least one cooling zone, not to operate any of the pumps of the at least one warming zone and the at least one cooling zone or to operate all pumps of the at least one warming zone and the at least one cooling zone at the reduced rated power so as not to dispense the treatment medium by the respective spraying apparatuses.

13. The thermal container treatment apparatus of one of claim 12, wherein the temperature sensor is provided in the tank below a filling level of the treatment medium or wherein the temperature sensor is provided in a return line.

14. The thermal container treatment apparatus of one of claim 1, wherein the temperature sensor is provided in the tank below a filling level of the treatment medium or wherein the temperature sensor is provided in a return line.

15. A method for controlling a thermal container treatment apparatus including a conveying apparatus for containers, a treatment zone with a spraying apparatus for dispensing treatment medium to containers, a tank for collecting and providing the treatment medium, a line connection between the tank and the spraying apparatus, wherein a pump is provided in the line connection, and a temperature sensor provided for measuring a temperature of the treatment medium, the method comprising:
operating, during normal operation in the treatment zone, a control apparatus to operate the pump at a rated power for dispensing the treatment medium by the spraying apparatus; and
operating, during standby operation in the treatment zone, the control apparatus not to operate the pump or to operate the pump at a reduced rated power without the treatment medium being able to be dispensed by the spraying apparatus.

16. The method of claim 15, wherein the line connection further comprises a connection to at least one temperature control valve, wherein the at least one temperature control valve is connected to a heating and/or cooling system for providing temperature-controlled treatment medium, and
the method further comprising controlling the at least one temperature control valve for controlling a temperature of the treatment medium in the tank.

17. The method of claim 16, wherein the thermal container treatment apparatus includes a plurality of the treatment zones, wherein the plurality of treatment zones is configured as warming zones or as cooling zones, and
the method further comprising:
during the standby operation of the thermal container treatment apparatus, not operating any pumps or operating all pumps at the reduced rated power so as not to dispense the treatment medium by the respective spraying apparatus.

18. The method of claim 15, wherein the thermal container treatment apparatus includes a plurality of the treatment zones, wherein the plurality of treatment zones is configured as warming zones or as cooling zones, and
the method further comprising:
during the standby operation of the thermal container treatment apparatus, not operating any pumps or operating all pumps at the reduced rated power so as not to dispense the treatment medium by the respective spraying apparatus.

19. The method of claim 18, further comprising, in order to achieve the normal operation of the thermal container treatment apparatus after the standby operation of the thermal container treatment apparatus:
at a first given point in time before a planned supply of containers to at least one warming zone, operating the pumps of the at least one warming zone and at least one cooling zone at the rated power for dispensing the treatment medium from spraying apparatuses of the at least one warming zone and the at least one cooling zone, and thereafter
at a second given point in time before the planned supply of containers from the at least one warming zone to at least one pasteurization zone, operating a pump of the at least one pasteurization zone at the rated power for dispensing the treatment medium from the spraying apparatus of the at least one pasteurization zone.

20. The method of claim 19, further comprising, in order to achieve the standby operation of a thermal treatment plant after achieving the normal operation of the thermal treatment plant:
after containers have left the at least one pasteurization zone, not operating the pump of the at least one pasteurization zone or operating the pump of the at least one pasteurization zone at the reduced rated power so as not to dispense the treatment medium by the spraying apparatus of the at least one pasteurization zone, and, thereafter,
after containers have left the last of the at least one cooling zone, not operating any of the pumps of the at least one warming zone and the at least one cooling zone or operating all pumps of the at least one warming zone and the at least one cooling zone at the reduced rated power so as not to dispense the treatment medium by the respective spraying apparatuses.
